(19) United States
(12) Patent Application Publication
(10) Pub. No.: US 2021/0008180 A1
(43) Pub. Date: Jan. 14, 2021

(54) FORMULATIONS CONTAINING MUCIN-AFFECTING PROTEASES

(71) Applicant: MUCPHARM PTY LTD, Malvern (AU)

(72) Inventors: David MORRIS, Malvern (AU); Sarah VALLE, Malvern (AU); Javed AKHTER, Malvern (AU); Krishna PILLAI, Malvern (AU)

(21) Appl. No.: 16/975,058

(22) PCT Filed: Feb. 22, 2019
(86) PCT No.: PCT/AU2019/050154

§ 371 Date: Aug. 21, 2020

(30) Foreign Application Priority Data
Feb. 23, 2018 (AU) 2018900588

(51) Int. Cl. A61K 38/48; A61K 9/50; A61P 35/00

(52) U.S. Cl. CPC A61K 38/4873; A61P 35/00; A61K 9/50

(57) ABSTRACT

Disclosed herein is a microsphere for delivery to a target area in a patients body. The microsphere contains a mucin-affecting protease loaded therein and is adapted to release the mucin-affecting protease in a sustained manner when exposed to physiological conditions. Also disclosed are pharmaceutical compositions comprising the microspheres and methods of treatment involving the microspheres.

### TECHNICAL FIELD

The present invention relates to microspheres containing mucin-affecting proteases loaded therein. In one form, the present invention relates to microspheres containing mucin-affecting proteases such as Bromelain, for use in treating mucin-producing cancers and other diseases involving mucin.

### BACKGROUND ART

Mucins are a family of high molecular weight, heavily glycosylated proteins produced by epithelial tissues, including those in the gastrointestinal tract, lungs, kidneys, ovaries, breast, and pancreas. Under normal physiological conditions, mucin plays a protective role for epithelial tissues. However, mucins can also be involved in a number of diseases. For example, overexpression of specific types of mucins (e.g. MUC1, MUC2, MUC4, MUC5AC, MUC5B, MUC16 and other mucins), are associated with some types of cancer. The synthesis of mucin on the surface of epithelial cells is normally highly regulated but mucin production is increased in tumours, partly due to an increased expression of human mucin. Mucin expression and composition is altered in cancers of epithelial origin, and mucus production is known to be a negative prognostic factor for patients affected by such cancers.

Abnormal accumulations of mucins can also deleteriously affect a patient's health, causing non-cancerous diseases such as cystic fibrosis and chronic obstructive pulmonary disease.

There is therefore a need to treat diseases involving mucin and provide better outcomes for patients suffering from such diseases. Mucin-related diseases can, for example, be treated with mucolytic agents, which are agents that affect (e.g. by breaking down or otherwise disrupting) the mucin proteins, making them less viscous and hence more easily cleared by the body or easier to penetrate with cytotoxic drugs (e.g. in the case where the mucins surround a tumour).

A class of mucolytic agents are mucin-affecting proteases, which are proteolytic enzymes that cause proteolysis of the mucin proteins. The effective delivery of mucin-affecting proteases into a patient may, however, be difficult because of the typically complex nature of the proteases and attendant risk of side effects. Mucin-affecting proteases can also have stability issues under physiological conditions.

For example, Bromelain is a mucin-affecting protease. Bromelain is an extract of the pineapple plant (Ananas Comosus) and contains various thiol proteases. Bromelain has proteolytic activity in vitro and in vivo, as well as antiedematous, anti-inflammatory, antithrombotic and fibrinolytic activities, and may therefore be used to treat conditions such as deep vein thrombosis and blood coagulation disorders. Bromelain has also shown anti-cancer properties in in vitro and in vivo models against certain types of cancers, both on its own and in combination with other chemotherapeutic agents.

Bromelain has therefore been proposed as a therapeutic drug for the treatment of certain types of cancers and other mucin-involving diseases. Clinical trials involving the systemic administration of Bromelain have, however, not yet been conducted due to risks (in particular its fibrinolytic action and effect on bleeding) associated with the systemic administration of therapeutically effective amounts of Bromelain (as seen in previous animal studies).

It would be advantageous to deliver therapeutically effective amounts of mucin-affecting proteases (such as Bromelain) to a patient in a manner whereby any potential side effects are minimised.

### SUMMARY OF INVENTION

In a first aspect, the present invention provides a microsphere for delivery to a target area in a patient's body. The microsphere contains a mucin-affecting protease loaded therein and is adapted to elute the mucin-affecting protease in a sustained manner when exposed to physiological conditions.

The loading of specific drugs into microspheres for local delivery into a patient's body is known, for example, in a technique known as transarterial chemoembolization (TACE). Microspheres sold under the brand name DC Bead® by Biocompatibles UK Ltd are, for example, indicated for the intra-arterial delivery of the anti-cancer agents Doxorubicin and Irinotecan for the treatment of primary and secondary liver cancers. The drugs described as being loadable into DC Beads® for sustained release are, however, all positively charged and relatively small (ca 600 Da) molecules, and drugs other than Doxorubicin and Irinotecan are described as not being able to be held within the microsphere appropriately. Indeed, even if loadable into the microspheres, many drugs are almost instantaneously released (commonly referred to as a “Burst release”) under physiological conditions. Other microspheres (some of which are described below) are similarly indicated only for use with small molecules such as Doxorubicin and Irinotecan.

In stark contrast, mucin-affecting proteases are enzymes (or enzymatic mixtures) having high molecular weights. In the case of Bromelain, for example, some enzymes have a reported molecular weight of around 33,000 Da. It is therefore not at all in accordance with the teachings of the prior art that mucin-affecting proteases such as Bromelain might be loadable into microspheres such as DC Beads® and, even more so, that the so-loaded proteases would subsequently be released in an active form from the microspheres, and at a sustained rate, under physiological conditions. Indeed, previous attempts by the present inventors and others to load Bromelain into microspheres have not been successful. In some of these attempts, for example, the Bromelain simply would not load into the microspheres. In other attempts, the Bromelain was found to decompose the microspheres, either resulting in the Bromelain itself degrading under ambient conditions or resulting in a “burst release” of the Bromelain following exposure to physiological conditions (which would have the same effect as if it had been systemically delivered).

As a result of these teachings of the prior art, it was generally thought that microspheres such as those described herein would not be useful for the sustained delivery of non-indicated molecules, let alone the sustained delivery of still-active large enzymes or enzymatic mixtures containing large enzymes.

The present invention has been made following the inventors' surprising and unexpected discovery that Bromelain (and subsequently Papain) can, in fact, be loaded into the microspheres (such as DC Beads®) described herein, and which are able to be locally delivered into a patient's body. Further, the so-loaded Bromelain has been surprisingly and unexpectedly found to elute from the microspheres in a still-active form and at a sustained rate when exposed to physiological conditions. The prolonged activity of Bromelain was a surprise to the inventors, given its usual instability at ambient conditions. Thus, the inventors have discovered that Bromelain can, contrary to conventional wisdom, be loaded into microspheres adapted to release the Bromelain in a sustained manner. The inventors' subsequent experiments have shown that Papain, another mucin-affecting protease, has comparable loading and elution properties to those of Bromelain. The inventors therefore believe that a reasonable prediction can be made that other mucin-affecting proteases will have utility in the present invention. Papain and ficin, for example, are similar in structure and function.

The inventors recognised that their discovery has the potential to provide a vehicle for the local delivery of a therapeutically effective amount of Bromelain and other mucin-affecting proteases, whereby their potential side effects are minimised. The significant advantages of this will be apparent to those skilled in the art and will be described in further below.

In a second aspect, the present invention provides a pharmaceutical composition comprising microspheres for delivery to a target area in a patient's body, the microspheres containing a mucin-affecting protease loaded therein and being adapted to elute the mucin-affecting protease in a sustained manner when exposed to physiological conditions; and a pharmaceutically acceptable carrier.

In a third aspect, the present invention provides a pharmaceutical composition comprising the microspheres of the first aspect of the present invention and a pharmaceutically acceptable carrier.

In a fourth aspect, the present invention provides a method for loading a mucin-affecting protease into microspheres. The method comprises adding the microspheres to a solution having an acidic pH (e.g. as low as 2 or 2.5); mixing the solution comprising the microspheres with a solution comprising the mucin-affecting protease; and agitating the mixture for a time sufficient for the mucin-affecting protease to be loaded into microspheres. Optionally, the solution to which the microspheres are added may have an ionic strength similar to that at a target area in a patient's body.

In a fifth aspect, the present invention provides a method for the treatment of a mucin-producing cancer, pseudomyxoma peritonei, cystic fibrosis or chronic obstructive pulmonary disease. The method comprises administering to a patient a therapeutically effective amount of microspheres containing a mucin-affecting protease loaded therein, wherein the microspheres are adapted to elute the mucin-affecting protease in a sustained manner following administration.

As noted above, microspheres loaded with Doxorubicin or Irinotecan are used in a process called transarterial chemoembolization (TACE) for the treatment of cancers such as non-resectable hepatocellular carcinoma. The microspheres are injected into an artery upstream of the tumour and form an embolus when the size of the artery decreases. The Doxorubicin or Irinotecan subsequently elutes from the microsphere very close to and directly into the blood vessel leading into the tumour, resulting in a high local concentration of drug. Such precisely targeted drug delivery can result in fewer drug-related adverse effects.

The inventors believe that microspheres in accordance with the present invention may be similarly effective for locally delivering mucin-affecting proteases when intra-arterially delivered into a patient. The inventors also expect that intralesional, intra-abdominal or intracavitary (e.g. into the peritoneum or pleural cavity) delivery of the microspheres will be similarly effective for treating other relevant diseases, as will be described below.

In a sixth aspect, the present invention provides a method for the treatment of a mucin-producing cancer, pseudomyxoma peritonei, cystic fibrosis or chronic obstructive pulmonary disease, comprising administering a therapeutically effective amount of the microspheres of the first aspect of the present invention or the pharmaceutical composition of the second or third aspect of the present invention to a patient in need thereof.

In a seventh aspect, the present invention provides a use of the microspheres of the first aspect of the present invention for the manufacture of a medicament for the treatment of a mucin-producing cancer, pseudomyxoma peritonei, cystic fibrosis or chronic obstructive pulmonary disease.

In an eighth aspect, the present invention provides a use of the microspheres of the first aspect of the present invention for the treatment of a mucin-producing cancer, pseudomyxoma peritonei, cystic fibrosis or chronic obstructive pulmonary disease.

In a ninth aspect, the present invention provides the microspheres of the first aspect of the present invention for use as a medicament.

In a tenth aspect, the present invention provides the microspheres of the first aspect of the present invention for use in the treatment of a mucin-producing cancer, pseudomyxoma peritonei, cystic fibrosis or chronic obstructive pulmonary disease.

In an eleventh aspect, the present invention provides a composition comprising microspheres into which a mucin-affecting protease has been loaded, the microspheres being adapted to elute the mucin-affecting proteas in a sustained manner when exposed to physiological conditions.

In a twelfth aspect, the present invention provides an injectable composition comprising microspheres into which a mucin-affecting protease has been loaded, the microspheres being adapted to elute the mucin-affecting protease in a sustained manner when exposed to physiological conditions.

In a thirteenth aspect, the present invention provides a sustained release formulation comprising microspheres into which a mucin-affecting protease has been loaded, the microspheres being adapted to elute the mucin-affecting protease in a sustained manner when exposed to physiological conditions.

Other aspects, features and advantages of the present invention will be described below.

### DETAILED DESCRIPTION OF THE INVENTION

As noted above, the present invention provides microspheres for delivery to a target area in a patient's body. The microspheres contain one (or more) mucin-affecting proteases loaded therein and are adapted to elute the protease(s) in a sustained manner when exposed to physiological conditions.

Intra-arterial delivery of microspheres is a relatively well established field, and biocompatible microspheres containing chemotherapeutics for local tumour delivery have been used in the treatment of certain tumours. For example, polyvinyl alcohol (PVA) hydrogel microspheres marketed under the brand name DC Bead® by Biocompatibles UK Ltd containing the chemotherapy agents Doxorubicin or Irinotecan (both positively charged small molecules) have been used for local tumour delivery using a technique known as transarterial chemoembolization (TACE) for treating primary and secondary liver cancers. These drug-eluting PVA hydrogel beads are also used with radioactivity, for example, selective internal radiation therapy (SIRT).

Following their surprising an unexpected discovery that Bromelain (and subsequently Papain) could be loaded into microspheres such as DC Beads® (and the other microspheres described below), experiments conducted by the inventors (described below) revealed that the Bromelain was even more surprisingly eluted from the microspheres in an active form and in a sustained manner under physiological conditions in vitro. The inventors believe that it is reasonable to predict from their preliminary experimental data that other types of microspheres will be capable of loading, containing and eluting Bromelain (and other mucin-affecting proteases) in a similar manner. Microspheres formed from substances which are biocompatible with patients' bodies and which will not adversely interact with the proteases contained therein are potentially useful in the present invention, and routine trials and experiments can be conducted to confirm whether or not a particular microsphere is adapted to elute the proteases contained therein in a sustained manner.

### Mucin-Affecting Proteases

As noted above, mucin-affecting proteases are a class of proteolytic enzymes which can cause proteolysis of mucin proteins and thereby provide a therapeutic effect. As used herein, the term “Mucin-affecting” is to be understood as affecting the mucin in any therapeutically effective manner such as, for example, liquefying or otherwise breaking-down the mucin (i.e. making it less viscous and more easily cleared by the body) or disrupting the mucin. Such proteases may be useful for the treatment of mucin-producing cancers (which, as defined below, may include mucin-secreting cancers and/or mucin-containing cancers and/or mucin-producing cancers) and other diseases involving mucin (e.g. as described below). The inventors believe that any mucin-affecting protease may be used in the present invention, with only routine trial and experimentation being required (in light of the teachings contained herein) in order to determine any particular mucin-affecting protease's suitability.

The present invention will primarily be described below in the context of Bromelain and Papain, both of which are plant-derived protease enzymes that affect mucin. A person skilled in the art would, however, appreciate that the teachings contained herein could be adapted, using routine trials and experiments, for any given mucin-affecting protease.

The mucin-affecting protease may, for example, be selected from one (or more) of the group consisting of mucin-affecting plant derived proteases, mucin-affecting fungal proteases and mucin-affecting bacterial proteases.

There are other plant-derived proteolytic enzymes that express the same characteristics as Bromelain and the inventors expect that any plant-derived protease enzymes which have a therapeutic effect on mucin (e.g. its production) may be used in the present invention, with routine experimentation be able to confirm the suitability of any particular plant-derived protease enzymes. In some embodiments, for example, the plant-derived protease enzymes may be selected from one or more of the group consisting of Bromelain, Papain (extracted from papaya), Ficain (extracted from figs), Actinidain (extracted from fruits including kiwifruit, pineapple, mango, banana and papaya), Zingibain (extracted from ginger) and Fastuosain (a cysteine proteinase from Bromelia fastuosa). Asparagus, mango and other kiwi fruit and papaya proteases may also be used.

The inventors also believe that mucin-affecting fungal proteases and mucin-affecting bacterial proteases may have similar utility in the present invention. Suitable fungal proteases may include aspergillus, serine proteases (subtilisin family), aspartic proteases (pepsin family) and metalloproteases (some of which are known to have anti-cancer activity by targeting the walls of epithelial cells). Suitable bacterial proteases may include those derived from silkworm peptizyme.

As used herein, the term Bromelain is to be understood to encompass one or more of the mucin-affecting and, optionally, otherwise therapeutically active substances present in the extract of the pineapple plant (Ananas Comosus). Bromelain is a mixture of substances (including different thiol endopeptidases and other components such as phosphatase, glucosidase, peroxidase, cellulase, esterase, and several protease inhibitors) and it may not be necessary for all of the substances contained in the extract be loaded into the microspheres, provided that the fraction of the substances loaded into the microspheres can at least affect mucin (e.g. by causing proteolysis of mucin proteins). The Bromelain used in the experiments described herein was commercially sourced from Challenge Bioproducts Co Ltd.

### Indications

The microspheres of the present invention may be delivered to a target area in the patient's body in order to treat any disease or condition for which the mucin-affecting proteases contained in the microsphere are efficacious. The microspheres, adapted to elute the mucin-affecting proteases loaded therein in a sustained manner once they are exposed to physiological conditions, can potentially be used to treat any diseases involving mucin and especially diseases where systemic delivery of the mucin-affecting proteases may be problematic.

For example, as noted above, Bromelain is known to have proteolytic activity in vitro and in vivo. Bromelain also has antiedematous, anti-inflammatory, antithrombotic and fibrinolytic activities, and has shown promise as an anti-cancer agent. However, Bromelain is not yet used as a clinical therapy for cancer due to the risks of its systemic administration, which may be problematic because of its fibrinolytic action and effect on bleeding. The local and sustained release of Bromelain achieved by the present invention, however, may potentially result in a high local concentration of Bromelain in a target area of a patient's body without the risk of systemic toxicity. The present invention also has the potential to improve the penetration of drugs into cancers and have a synergistic effect on the cytotoxicity of certain chemotherapy agents.

The present invention may, for example, provide for the treatment of mucin-producing cancers, pseudomyxoma peritonei, cystic fibrosis and chronic obstructive pulmonary disease. When the mucin-affecting protease has further therapeutic activity, then the present invention may also provide for the treatment of other conditions. In the case of Bromelain, for example, the present invention may also provide for the treatment of deep vein thrombosis and blood coagulation disorders.

Mucin-affecting proteases cause proteolysis of mucin proteins and will therefore affect (e.g. by breaking down) any mucin at the target area in the patient's body upon delivery thereto. At the very least, therefore, delivery of the microspheres of the present invention to the target area in the patient's body (e.g. a mucin-producing tumour) will cause the mucin in that area (e.g. surrounding the tumour) to be affected and thereby provide some therapeutic effect (e.g. a reduction in mass or improved circulation or digestion ability in the target area). Further, co-administered therapeutic agents (e.g. as will be described below) would be able to more effectively penetrate into the target area (e.g. tumour) than would be the case if the mucin had not been affected. As would be appreciated, this is a very useful therapeutic effect and may significantly increase the efficacy of existing treatment regimens, potentially allowing lower doses of the co-administered therapeutic agents to be used.

The present invention may be used for the treatment of mucin-producing cancers. As used herein, the term “Mucin-producing” cancers is to be understood to mean both cancers which contain mucin and cancers which produce mucin. Cancers that contain mucin include, for example, signet ring cell cancers and goblet cell cancers. Mucin can also be in the cytoplasm of the cell that isn't characterised as a signet ring or goblet cell. Cancers that produce mucin include, for example, the mucin secreting type, such as pseudomyxoma, or cancers that have an overexpression of mucin or secrete mucin around their cells (walls), which acts as a barrier to penetration of chemotherapy and also prevents immune cell recognition.

By way of example, cancers which produce mucin include lung cancer, liver cancer, pancreatic cancer, thyroid cancer, stomach cancer, cancer of the appendix, peritoneal cancer, hepatocellular cancer, prostate cancer, breast cancer, colorectal cancers, ovarian cancers, mesothelioma, neuroblastoma, small bowel cancer, lymphoma and leukaemia. Many of these cancers are difficult to treat with conventional therapies. The treatment of hepatocellular carcinoma (primary liver cancer), liver metastases (secondary liver cancer) and pancreatic cancer are particularly preferred applications of the present invention. The microspheres of the present invention may also be used to treat adenocarcinoma. In particular, the adenocarcinoma may be signet ring cell carcinoma. The microspheres of the present invention may also be used to treat pseudomyxoma peritonei.

Hepatocellular carcinoma (primary liver cancer) is commonly caused by hepatitis B and C infection, cirrhosis of any cause including alcohol, non-alcoholic steatohepatitis (NASH) and other less common causes. Current treatments include liver transplantation, resection and thermal ablation, but only a minority are treatable by these potentially curative procedures. The majority of patients receive TACE and microsphere delivery of doxorubicin, but this is limited in terms of response rate, and many patients still have a limited survival.

Liver metastases (secondary tumours) can occur from a variety of cancers including colorectal, stomach, pancreas and other adenocarcinomas and tumours from both abdominal and other sites in the body. Liver resection is the optimal therapy although, in selected cases, thermal ablation may now produce similar outcomes. Systemic chemotherapy is widely used with modest outcomes. Micro sphere delivery of Irinotecan has been used as a palliative treatment for liver metastases of colorectal origin.

Delivery of the microspheres of the present invention into the patient's tumour via a feeding artery (e.g. the hepatic artery, when treating liver cancers), where the sustained release of the mucin-affecting proteases will have maximum effect and reduced risk of the side effects associated with the systemic delivery, may provide a much less invasive procedure for the treatment of a liver tumour or pancreatic tumour, due to the mucin-affecting proteases being delivered at the target site.

Further, some mucin-affecting proteases may themselves have anti-cancer activity. Bromelain, for example, has been found to have anti-cancer activity against a number of cancers, including, for example, pancreatic cancer, hepatocellular cancer, prostate cancer, breast cancer, colorectal cancers, thyroid cancer, stomach cancer, cancer of the appendix, peritoneal cancer, hepatocellular cancer, mesothelioma, pseudomyxoma peritonei and other peritoneal cancers, ovarian cancer, lung cancer, small bowel cancer and other cancers. Papain, for example, may be used for treating cancers such as lung cancer, pancreatic cancer, liver cancer, ovarian cancer, neuroblastoma, lymphoma, leukaemia or other solid cancers. Thus, delivery of microspheres of the present invention containing Bromelain or Papain to a mucin-producing tumour will affect (e.g. disrupt or otherwise break down) the mucin surrounding the tumour and enable a more effective penetration of the Bromelain or Papain into the tumour, where its anti-cancer activity should be more efficacious (especially as it will be delivered in a sustained manner over a period of time).

Pseudomyxoma peritonei (PMP) is a form of tumour characterized by excessive accumulation of mucin, secreted by tumour cells, in the peritoneal cavity. The tumour cells are primarily of appendiceal origin although disseminated cancers of the colon, rectum, stomach, gall bladder, small intestines, urinary bladder, lungs, breast, pancreas and ovary may also contribute to the disease. The mucinous mass that is secreted accumulates in the abdominal cavity and causes increased internal pressure on the digestive tract, which is associated with significant morbidity and mortality due to nutritional compromise.

Traditionally, laparotomy, removal of mucinous mass and cytoreduction followed by hyperthermic intraperitoneal chemotherapy (HIPEC) has been the preferred treatment for PMP patients. Since the disease is progressive, however, patients may require several treatments during the course of the disease, which has the consequence of increased morbidity and even death.

Delivery of the microspheres of the present invention into the patient's peritoneum, where the sustained release of the mucin-affecting proteases will have maximum effect and reduced risk of the side effects associated with a systemic delivery, may provide a much less invasive procedure for the removal of mucinous mass, due to the mucin-affecting proteases liquefying the accumulated mucin (i.e. so that the body can more readily remove it, or the liquefied mucin can more easily be sucked out from the peritoneum) and also potentially treating the cancer (e.g. due to the anti-cancer activity of the protease or a co-administered chemotherapeutic, etc.).

The present invention may be used for the treatment of cystic fibrosis and chronic obstructive pulmonary disease. Cystic fibrosis is a disorder that damages the lungs and digestive system. It affects the cells that produce mucus, sweat and digestive juices by causing these fluids to become thick and sticky, whereupon they can plug up tubes, ducts and passageways. Chronic obstructive pulmonary disease (COPD) are a group of lung diseases (including emphysema and chronic bronchitis) that block airflow and make it difficult to breathe. It is expected that a localised and sustained delivery of the mucin-affecting proteases into the lungs of the patient may be an effective therapeutic regimen.

In some embodiments, specific mucin-affecting proteases may have therapeutic applications in addition to their mucin-affecting properties. Examples of such embodiments will be described below.

The formation of blood clots (thrombi) lies at the basis of a number of serious diseases such as myocardial infarction, coronary artery disease, stroke, massive pulmonary embolism and acute limb ischaemia. The likelihood of suffering thrombosis may also be increased in patients who are fitted with a stent. Anticoagulant drugs (such as heparin and warfarin) may be used to treat thrombosis. However, such anticoagulants only inhibit the formation of thrombi or inhibit the growth of existing thrombi. There is some evidence that proteases may generally reduce blood clotting when administered to a patient. In the case of proteases such as Bromelain, for example, such therapeutic effects are well documented. Thus, in embodiments of the present invention comprising Bromelain (at least) the microspheres may be useful in treating conditions such as deep vein thrombosis, blood coagulation disorders, haemophilia, myocardial infarction, coronary artery disease, stroke, massive pulmonary embolism and acute limb ischemia, stent-related thrombosis or haemarthrosis. Similar to that described above, localised delivery of the Bromelain to relevant area in the patient's body may be far more effective and involve fewer side effects than the systemic delivery of Bromelain.

Furthermore, synergistic effects may be obtained when the mucin-affecting proteases are used in combination with other therapeutically effective agents. For example, when Bromelain is used in combination with another mucolytic agent (as will be described in further detail below), the microspheres of the present invention may be even more efficacious in treating other diseases involving mucin, such as glue ear, sputum retention, chest infection and mucus and cellular debris associated with biliary/pancreatic stents.

As also described herein, the use of mucin-affecting proteases, such as Bromelain for example, in combination with another chemotherapeutic agent or agents can also result in a synergistic effect, with the Bromelain (for example) facilitating the chemotherapeutic agent(s) entry into (or deeper into) the tumour. As would be appreciated, such a mechanism has the potential to improve the efficacy of the chemotherapeutic agent(s), and potentially at lower dosages.

### Microspheres

The microspheres of the present invention may take any suitable form and be formed from any suitable biocompatible substance or combination of substances, provided that the mucin-affecting proteases can be loaded therein (and contained for therapeutically relevant periods of time without significantly adversely affecting their activity), be deliverable to a target area in the patient's body and elute the proteases in a sustained manner when exposed to physiological conditions (i.e. once at the target area).

The microspheres may retain the mucin-affecting proteases therein using any suitable mechanism. In some embodiments, for example, the chemical charge or functional groups in the microspheres may be sufficient to retain the proteases. Alternatively (or in addition), steric effects (e.g. pore size) may be sufficient to retain the proteases in the microspheres until exposure to physiological conditions. Similarly, the microspheres may elute the mucin-affecting proteases using any suitable mechanism. In some embodiments, for example, the proteases may leech out of pores in the microspheres under physiological conditions. In some embodiments, the microspheres may themselves biodegrade under physiological conditions, with the proteases being released at a sustained rate as the microspheres degrade. In some embodiments, exposure of the microspheres to physiological conditions may cause the chemical factors (e.g. the chemical charge or functional groups in the microspheres) to change such that the microspheres no longer retain the proteases such that the proteases are released at a sustained rate. In some embodiments, exposure of the microspheres to physiological conditions may cause the microsphere's pores to enlarge such that the proteases are released at a sustained rate.

Based on the factors described in the preceding paragraph and the teachings contained herein, the inventors believe that a reasonable prediction can be made regarding whether or not a particular microsphere will be useful in the present invention with a particular mucin-affecting protease. Routine experiments, such as those described below (adapted accordingly), can be performed in order to confirm this prediction.

The microspheres may, for example, comprise (or be defined by) a matrix into which the protease enzymes are loadable. Bringing the microspheres and mucin-affecting proteases into contact with each other under appropriate conditions (e.g. as described below) results in the proteases becoming incorporated into the matrix and hence loaded into the microsphere.

Although the inventors envisage that the microspheres would usually be purchased from commercial sources (which already have approval for human therapeutic use), it may also be possible for the microsphere to be formed from a composition that includes the proteases. In such embodiments, the matrices would form around the enzymes, which may provide a more homogeneous dispersion of the enzymes throughout the so-formed microspheres and result in a longer lasting sustained release of the enzymes from the microspheres at the target site.

The microsphere may, for example, comprise (or be defined by) a hydrogel into which the mucin-affecting proteases are loadable. One suitable hydrogel is a polyvinyl alcohol (PVA) hydrogel. Specific microspheres formed from a PVA hydrogel and trialled by the inventors are the commercially available biocompatible polyvinyl alcohol (PVA) hydrogel microspheres marketed under the brand name DC Bead® by Biocompatibles UK Ltd. These microspheres are produced from a polyvinyl alcohol (PVA) hydrogel that has been modified with sulphonate groups, and have previously been used for the controlled loading and delivery of the chemotherapeutic drugs Doxorubicin or Irinotecan and used in trans-arterial chemoembolization (TACE). Variations on the commercially available DC Beads® are described, for example, in WO 2001/68722 entitled “Hydrogel biomedical articles”, the contents of which are hereby incorporated by reference. DC Beads® are sold in a range of sizes, having size ranges 70-150 μm, 100-300 μm, 300-500 μm and 500-700 μm

Another suitable hydrogel is a poly(vinyl alcohol-co-sodium acrylate) hydrogel. Specific microspheres formed from a poly(vinyl alcohol-co-sodium acrylate) hydrogel and trialled by the inventors are the commercially available microspheres marketed under the brand name HepaSphere™ Microspheres. HepaSphere™ Microspheres are made from vinyl acetate and methyl acrylate and in an acidic environment. Anticancer drugs such as Doxorubicin are loadable into the HepaSphere™ Microspheres, with the microspheres being indicated for delivery into the patient via the TACE procedure described above. HepaSphere™ Microspheres are sold in a range of sizes, having size ranges 30-60 μm, 50-100 μm, 100-150 μm and 150-200 μm.

Another suitable hydrogel is a hydrogel core consisting of sodium poly(methacrylate) and an outer shell of poly(bis[trifluoroethoxy]phosphazene). Specific microspheres formed from this hydrogel and trialled by the inventors are the commercially available micro spheres marketed under the brand name Embozene TANDEM™, marketed by Boston Scientific. Similar to the preceding microspheres, doxorubicin-HCl or irinotecan-HCl are loadable into the Embozene TANDEM™ Microspheres for use in the TACE procedure. Embozene TANDEM™ are sold in sizes 40±10 μm, 75±15 μm or 100±25 μm.

As noted above, the molecules described as being loadable into DC Beads®, HepaSphere™ Microspheres and Embozene TANDEM™ Microspheres for sustained release are, however, all positively charged and relatively small (ca 600 Da) molecules, and drugs other than such are described as not being able to be held within the microsphere appropriately. It is therefore completely surprising that proteolytic enzymes such as Bromelain and Papain can be loaded into, stably contained therein and subsequently eluted in a sustained manner from these microspheres.

Other commercially-available microspheres of which the inventors are aware and believe would be suitable for use with the present invention include those sold under the brand LifePearl by Terumo Europe NV. These microspheres consist of a hydrogel network of poly(ethylene glycol) and 3-sulfopropyl acrylate. The inventors also believe that microspheres formed from poly(lactic-co-glycolic acid) (PLGA) and polylactic acid (PLLA) hydrogel networks would be suitable for use with the present invention.

In some embodiments the microsphere may comprise an outer coating, where such a coating may impart beneficial properties to the microsphere. For example, it may be beneficial to coat the microsphere with a coating that must first be dissolved (or otherwise removed) before the protease enzymes can begin to elute. In this manner, for example, the microspheres have time to reach the tumour site (for example) post-delivery before the enzymes start to elute. It might also be beneficial to coat the microsphere with a coating that protects the microsphere post-delivery and until such time as appropriate physiological conditions are reached (e.g. the pH and ion concentration at the target area).

The inventors have found, for example, that microspheres comprising an alginate outer coating can delay the start of the sustained release of the protease enzymes post exposure to physiological conditions. Other coating agents, such as those comprising chitosan, may also be useful in the present invention.

The inventors also expect that glass, resin and ceramic microspheres may have utility in the present invention. For example, glass microspheres marketed under the brand TheraSphere® are indicated in some countries as a radiotherapy treatment for hepatocellular carcinoma (HCC). The radioactive glass microspheres (20-30 micrometres in diameter) are infused into the arteries that feed liver tumours, where they embolize in the liver's capillaries and bathe the malignancy in high levels of yttrium-90 radiation. The inventors believe that TheraSphere® glass microspheres may be adaptable to contain mucin-affecting proteases for use in accordance with the teachings of the present invention. Similarly, ceramic microspheres, such as those marketed under the brand Ceramispheres, or resin microspheres such as those marketed under the brand SIR-spheres®, may be adaptable to contain mucin-affecting proteases for use in accordance with the teachings of the present invention.

In some embodiments, it is envisaged that different microspheres may be combined for co-administration to the patient. The different microspheres may contain the same or different mucin-affecting proteases or, indeed, any other active agents, such as those described below. The different microspheres may differ in respect of their size, their size distribution and/or their composition.

In order to be useful in the present invention, the microspheres should ideally be generally spherical and of a size in the micrometre range. Spherical microspheres are suitable for embolization, for example, since they offer less resistance to flow when delivered through blood vessels. Furthermore, spherical particles having a certain dimension can provide a higher density of particles within a specific volume.

The microspheres may have any size in the microsphere range (measured at their diameter), with the size of the microspheres useful in specific applications being dependent on factors such as the nature and quantity of the mucin-affecting proteases loaded therein (e.g. greater quantities of protease will require larger amounts of microspheres), the microspheres' delivery route into the patient (e.g. in embolization-related treatments, the size of the blood vessels at which embolization is to occur will govern the necessary size of the microspheres). As would be appreciated, there will always be a range of diameters in a batch of microspheres.

In some embodiments, for example, the microspheres may have a diameter of between about 30 and about 700 μm, although diameters of up to just under 1000 μm may be appropriate for peritoneal delivery and applications. In some embodiments, for example, the microspheres may have a diameter of between about 30 and about 500 μm, between about 50 and about 400 μm, between about 60 and about 300 μm, between about 80 and about 200 μm, between about 60 and about 100 μm, between about 50 and about 100 μm, between about 40 and about 80 μm, between about 30 and about 60 μm, between about 30 and about 50 μm or between about 40 and about 100 μm. In some embodiments, for example, the microspheres may have a diameter of about 700 μm, about 600 μm, about 500 μm, about 400 μm, about 300 μm, about 200 μm, about 100 μm, about 80 μm, about 70 μm, about 60 μm, about 50 μm, about 40 μm or about 30 μm.

Generally speaking, the larger microspheres will be more useful for delivery via intracavitary routes (e.g. intraperitoneally, to treat PMP or other peritoneal cancers), where greater quantities of mucin-affecting proteases (and potentially other active agents) would be beneficial. The smaller microspheres will generally be more useful in intra-arterial delivery routes, where they can flow through the artery until they embolise at the target area.

The microspheres of the present invention are adapted such that the mucin-affecting proteases are eluted in a sustained manner upon delivery to the target area. The mechanism via which the proteases are eluted is not important, so long as the elution is at a sustained rate. As noted above, the microspheres may, for example, include a number of pores through which the enzymes can elute. In some embodiments, the microsphere may itself degrade under physiological conditions, thereby exposing the loaded proteases.

As used herein, the phrase “in a sustained manner” is to be understood to mean that the mucin-affecting protease(s) contained within the microsphere elute over a therapeutically beneficial length of time. Whilst there will often be a “Burst release”, where a certain proportion of the loaded proteases rapidly elute when the microspheres are first exposed to physiological conditions, the rate elution of the proteases then slows down such that the remainder of the proteases contained in the microsphere elutes over a timeframe of a few hours, days or perhaps even weeks. The rate of release of the proteases need not be consistent over the whole of the elution period.

The rate at which, and the time period over which, the proteases elute from the microsphere may be varied depending on the specific application. Typically, however, the proteases should ideally be released for at least as long as the time over which the cells in the target area take to replicate. In this manner, the proteases (as well as any other active agents contained within the microsphere) are likely to be present to inhibit cell replication, leading to cell death.

It would usually also be necessary to take into account the rate at which the proteases will be cleared post-delivery to the target area and elution. For example, if delivered to areas having a relatively high blood flow therethrough, it would be expected that the proteases would be cleared more quickly than would be the case in areas having relatively low blood flow (noting, however, that the flow may be significantly hindered by the embolism). The release rate of the proteases from the micro sphere may need to be adapted to take such into account.

In a specific embodiment, for example, the mucin-affecting proteases may be released from the microspheres post-delivery to the target area in a sustained manner and over a period of time of up to about 120 hours, or possibly even longer. In some embodiments, for example, the mucin-affecting proteases may be released from the microspheres over a period of time of between about 10 hours to about 120 hours, between about 20 hours to about 100 hours, between about 30 hours to about 80 hours, between about 10 hours to about 50 hours, between about 15 hours to about 40 hours, between about 10 hours to about 30 hours or between about 10 hours to about 20 hours.

The microspheres of the present invention may (subject to loading and size constraints) contain any amount of the mucin-affecting proteases that can provide a therapeutic effect for the relevant condition. The amount of proteases able to be loaded into a particular microsphere would usually need to be empirically determined on a case by case basis, as will its release profile. The quantity of the mucin-affecting proteases initially loaded into the microspheres and subsequently delivered into the patient's body will depend on a number of factors, including the nature of the condition being treated, the sustained release rate of the proteases and the period of time over which the proteases need to be released.

In some embodiments, it may be necessary to deliver a relatively greater amount of microspheres in order to obtain a particular release profile of the proteases and/or quantity of the proteases delivered. In the case of Bromelain, for example, microspheres having a diameter of 300-500 μm may be loaded with as much as about 1800 μg Bromelain in 60 μL of the microspheres. For example, for treating tumours within a certain locality with Bromelain, the characteristics and dimension of the tumours will be a primary factor affecting the quantity of Bromelain-loaded microspheres required.

The microspheres of the present invention are capable of being delivered to a target area in the patient's body. Any method of delivery via which the microspheres arrive at the target area substantially intact and having lost as little as possible of the mucin-affecting proteases (etc.) contained therein may be used in the present invention.

Typically, the microspheres are adapted to be delivered locally to the target area (which will depend primarily on the disorder that is to be treated). Such a local delivery ensures that the maximum number of microspheres (and hence mucin-affecting proteases) are delivered to the area in the body where they are needed, which will maximize the efficacy of the treatment and minimise its potential side effects. The microspheres may, for example, be adapted to be delivered to the patient intra-arterially, intra-lesionally, intra-abdominally or intracavitarily (e.g. into the patient's peritoneum or pleural cavity). Other intracavitary delivery routes include intranasal and intrabronchial (which may be useful if treating cystic fibrosis, etc.), into the cavity of the bladder, or into the bile ducts (e.g. for cholangiocarcinoma).

The target area in the patient's body may be a tumour. The tumour may, for example be located in the patient's abdomen (e.g. in their pancreas, liver, colon, ovary or prostate). The tumour may, for example be located in the patient's lung. Similar to the transarterial chemoembolization (TACE) process described above, the microspheres may be administered into the feeding vessels of such a tumour to achieve high local concentrations of the mucin-affecting proteases over a sustained period. Subsequent doses of the microspheres may be delivered if sustained release over an even longer period of time would be beneficial for treatment.

Alternatively, the microspheres of the present invention may be delivered by intraperitoneal injection if treating pseudomyxoma peritonei or other peritoneal cancers. As noted above, larger microspheres can be delivered into cavities such as the patient's peritoneum, meaning that larger doses of the proteases (etc.) can be administered.

Alternatively, the microspheres of the present invention may be delivered by injection at the site of the thrombus when treating thrombi such as deep vein thrombosis.

The microspheres of the present invention are adapted to elute the proteases in a sustained manner when exposed to physiological conditions. As will be appreciated, different administration regimes will result in the microspheres being delivered into different parts of the patient's body (e.g. into an artery or into a cavity), which may expose them to different physiological conditions. For example, whilst the temperature throughout a patient's body would likely be reasonably consistent, the pH and electrolyte concentrations (for example) may differ between their arteries and cavities. It is within the ability of a person skilled in the art to assess these parameters (by pre-testing, if necessary) in order to adapt the microspheres of the present invention accordingly.

### Further Agents

Although efficacious on their own (i.e. due to their effects on diseases involving mucin, as described above), the mucin-affecting proteases contained within the microspheres of the present invention may also be used in in combination with further agents. Examples of such further agents are described below. When needed (or beneficial), the quantities of such further agents may be determined on an as-needed basis using no more than routine trials and experimentation.

The microspheres may themselves contain the further agent in addition to the mucin-affecting protease (i.e. the proteases and further agent are co-loaded). Alternatively, the further agent may be delivered to the patient (and hence the target area) in combination with the microspheres (administered together or separately (e.g. sequentially, in any order) and via the same or different routes). The further agent may, for example, be present in a carrier for the microspheres, or chemically bound to a surface of the microspheres. Alternatively, or in addition, the further agent may be contained in separate microspheres from those containing the mucin-affecting proteases, with both sets of microspheres being delivered in combination to the patient (either before, after or simultaneously). Microspheres containing mucin-affecting proteases such as Bromelain delivered locally to a tumour may, for example, also be combined with systemic chemotherapy regimens (i.e. where the chemotherapeutic agent is delivered orally or intravenously). The further agent may, in some embodiments, be systemically delivered (e.g. orally or IV) before, during or after delivery of the microspheres.

The further agent may, for example, be selected from one or more of the group consisting of a chemotherapeutic agent, a radiotherapeutic agent, another mucolytic agent and a contrast agent. Each of these further agents will be described in more detail below.

A chemotherapeutic agent is a pharmacologic agent for use in the treatment of cancer. Examples of chemotherapeutic agents which may be useful in the context of the present invention are listed in WO 2014/094041, the contents of which are herein incorporated by reference. Specific chemotherapeutic agents that may be used in the present invention may, for example, include gemcitabine, paclitaxel, docetaxel, doxorubicin, irinotecan, mitomycin C, oxaliplatin, carboplatin, 5-fluorouracil (or similar) and/or cisplatin. The inventors have previously described the desirable synergistic effects observed when some of these chemotherapeutic agents are co-administered with Bromelain and a mucolytic agent, and it is envisaged that these effects may also be utilised in the present invention. Doxorubicin, gemcitabine, 5-fluorouracil, mitomycin C, paclitaxel, Taxol, oxaliplatin and cisplatin in particular, have all been observed by the inventors to exhibit synergistic properties with Bromelain.

For example, as described above, the administration of Bromelain in micro spheres intra-arterially is expected to increase the efficacy of chemotherapy, whether the chemotherapeutic agent(s) are delivered systemically, co-loaded in the same spheres, or in separate spheres (administered at the same time or sequentially). The inventors believe that the addition of bromelain to microspheres delivered at the target area will provide an alternative treatment for hepatocellular carcinoma or primary liver cancer, liver metastases and pancreatic cancer and may potentially increase the anti-tumour effect of doxorubicin and other chemotherapies.

A radiotherapeutic agent may also be co-delivered with the microspheres containing the mucin-affecting proteases, for example, in order to show site of delivery and/or to increase the efficacy of the mucin-affecting proteases. Bromelain, for example, is a known PARP inhibitor and its co-administration with radiation may hinder the repair of DNA which is damaged by the radiation, resulting in localised cell death.

Whilst the radiotherapeutic agent might in theory be co-loaded into the microsphere carrying the proteases, it would need to be established that doing so would not cause damage to the proteases and affect their therapeutic activity. More likely, the radiotherapeutic agent would be separately delivered to the patient from the protease-containing microspheres, where any radiation damage to the mucin-affecting proteases would be minimised.

The radiotherapeutic agent may, for example, be separately provided in glass, resin or ceramic spheres, such as those commercially available under the brands QuiremSpheres® and SIR-Spheres® Y-90 resin microspheres. Alternatively (or in addition), radiotherapy may be co-delivered by external beam radiotherapy or brachytherapy, both of which can sensitise tumours.

As noted above, mucolytic agents affect (e.g. by disrupting or dissolving, etc.) mucus and are presently used to help relieve respiratory difficulties. Whilst mucin-affecting proteases are a class of mucolytic agent, in the context of the present invention the mucolytic agent described herein is defined to be a non-enzymatic agent which is distinct from the mucin-affecting proteases. The combination of such a mucolytic agent with the mucin-affecting proteases can provide advantages, some of which are described herein.

In WO 2014/094041, some of the present inventors described the beneficial effects of Bromelain when administered in conjunction with mucolytic agents (such as N-acetylcysteine) and chemotherapeutic agents. The combination of Bromelain and a mucolytic agent was found to significantly increase the effect and cytotoxicity of chemotherapy agents in mucin producing cancer cells, to have a direct anti-tumour effect and inhibitory effect on cancer cell viability and growth, to profoundly affect tumour-production of mucin, and be highly effective in liquefying tumour mucin. Benefits of Bromelain included increased penetration of chemotherapy into a cancer cell, increased penetration of chemotherapy into tumour stroma and synergy with certain chemotherapeutic agents. Bromelain also has tumour entry advantages especially in tumours with fibrous coats or which are surrounded in adhesions.

In WO 2017/063023, the contents of which are herein incorporated by reference, some of the present inventors described the surprising and unexpected synergistic effects of Bromelain in conjunction with the mucolytic agent cysteamine (or a metabolite, pharmaceutically acceptable salt, solvate or prodrug thereof). This combination was found to be highly effective when treating solid or hard tumours.

When they contain a mucolytic agent, the microspheres of the present invention may be even more efficacious in treating diseases involving mucin, such as mucin-producing cancers, pseudomyxoma peritonei, glue ear, cystic fibrosis, sputum retention, chest infection and mucus and cellular debris associated with biliary/pancreatic stents, as well as diseases involving thrombi such as haemophilia, myocardial infarction, coronary artery disease, stroke, massive pulmonary embolism and acute limb ischaemia, stent-related thrombosis or haemarthrosis (as described above). Whilst these conditions are treatable using mucin-affecting proteases alone, the efficacy of the treatment may be improved by co-administering a further mucolytic agent.

The mucolytic agent may, for example, be a thiol-containing mucolytic agent that reduces or disrupts disulphide bonds in mucins. Specific examples of mucolytic agents include N-acetyl cysteine (“NAC”), cysteamine, nacystelyn, mercapto-ethanesulphonate, carbocysteine, N-acystelyn, erdosteine, dornase alfa, gelsolin, thymosin P4, dextran and heparin. NAC is also an antioxidant and antigenotoxic agent and its safety in high doses for long periods is well established in human patients, primarily for respiratory disease. Other mucolytic agents are described in WO 2014/094041 and WO 2017/063023, and are hereby incorporated by reference.

A contrast agent may also be contained in the microspheres, for example if it would be advantageous to be able to be able to detect the location of the microspheres post-delivery or to determine the correct site of administration. Such fluorescence may assist in visually identifying the correct site and assist with dose distribution.

### Methods of Forming Microspheres

The present invention also provides a method for loading mucin-affecting proteases into microspheres. The method comprises adding the microspheres to a solution having an acidic pH and, optionally, an ionic strength similar to that at a target area in a patient's body; mixing the solution comprising the microspheres with a solution comprising the mucin-affecting proteases; and agitating the mixture for a time sufficient for the mucin-affecting proteases to be loaded into microspheres.

The inventors have discovered that the pH at which the mucin-affecting proteases are loaded into the microspheres can affect the quantity which can be loaded and can subsequently affect the rate of release of the enzymes upon exposure to physiological conditions. In the case of Bromelain, for example, lowering the pH has been found to cause better loading into microspheres and a slower release rate post-delivery to the target area. Without wishing to be bound by theory, the inventors' speculate that this effect may be due to the nett charge on Bromelain increasing at lower pH and/or that lowering the pH affects the pore size and hence the release pattern of the microspheres.

The inventors' preliminary experiments have shown that loading mucin-affecting proteases in the form of Bromelain at a pH as low as 2 or 2.5 can be beneficial in this regard.

Similarly, the inventors have discovered that the loading medium in which the mucin-affecting proteases are loaded into the microsphere can subsequently affect the rate of release of the enzyme upon exposure to physiological conditions.

As a general rule, the inventors have found that loading media having an acidic pH and ion concentration similar to that expected at the target area in the patient's body result in good loading into the microsphere and subsequent release at a sustained rate. Specific examples for loading Bromelain and Papain into specific microspheres are described in more detail in the Examples.

### Pharmaceutical Compositions

The present invention also provides pharmaceutical compositions comprising:

microspheres (e.g. the microspheres described above) for delivery to a target area in a patient's body, the microspheres containing mucin-affecting proteases loaded therein and being adapted to elute the proteases in a sustained manner when exposed to physiological conditions; and

a pharmaceutically acceptable carrier.

The pharmaceutically acceptable carrier for use in the pharmaceutical compositions of the present invention will depend on the route of administration of the composition. Liquid form preparations may include solutions, suspensions and emulsions, for example water or water-propylene glycol solutions for parenteral injection or intraperitoneal administration or injection. Suitable pharmaceutically acceptable carriers for use in the pharmaceutical compositions of the present invention include physiologically buffered saline, dextrose solutions and Ringer's solution, etc.

Liquid form preparations and aerosol preparations including the microspheres of the present invention may also be useful for intranasal administration, for example in treating cystic fibrosis. Aerosol preparations suitable for inhalation may, for example, include solutions and solids in powder form, which may be in combination with a pharmaceutically acceptable carrier, such as an inert compressed gas, e.g. nitrogen.

Pharmaceutical compositions suitable for delivery to a patient may be prepared immediately before delivery into the patient's body, or may be prepared in advance and stored appropriately beforehand.

The pharmaceutical compositions and medicaments of the present invention may comprise a pharmaceutically acceptable carrier, adjuvant, excipient and/or diluent. The carriers, diluents, excipients and adjuvants must be “acceptable” in terms of being compatible with the other ingredients of the composition or medicament and the delivery method, and are generally not deleterious to the recipient thereof. Non-limiting examples of pharmaceutically acceptable carriers or diluents which might be suitable for use in some embodiments are demineralised or distilled water; saline solution; vegetable based oils such as peanut oil, safflower oil, olive oil, cottonseed oil, maize oil; sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxylpropylmethylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from about 10% to about 99.9% by weight of the composition or medicament.

It will be understood that, where appropriate, some of the components in the microspheres or pharmaceutical compositions of the present invention may also be provided in the form of a metabolite, pharmaceutically acceptable salt, solvate or prodrug thereof.

“Metabolites” of the components in the microspheres of the invention refer to the intermediates and products of metabolism.

“Pharmaceutically acceptable”, such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base addition salts that retain the biological effectiveness and properties of the components and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluene sulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. The chemical modification of a pharmaceutical compound (i.e. drug) into a salt is a technique well known to pharmaceutical chemists to obtain improved physical and chemical stability, hygroscopicity, flow ability and solubility of compounds. See, e.g., H. Ansel et. al., Pharmaceutical Dosage Forms and Drug Delivery Systems (6th Ed. 1995) at pp. 196 and 14561457, which is incorporated herein by reference.

“Prodrugs” and “solvates” of some components in the microspheres or pharmaceutical compositions of the invention are also contemplated. The term “prodrug” means a compound (e.g., a drug precursor) that is transformed in vivo to yield the compound required by the invention, or a metabolite, pharmaceutically acceptable salt or solvate thereof. The transformation may occur by various mechanisms (e.g., by metabolic or chemical processes). A discussion of the use of prodrugs is provided by T. Higuchi and W. Stella, “Prodrugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

### Methods of Treatment

The present invention also provides methods for the treatment of diseases and conditions involving mucin, against which mucin-affecting proteases have a therapeutically relevant activity. For example, Bromelain has therapeutically relevant activity for treating mucin-producing cancers, pseudomyxoma peritonei, cystic fibrosis, chronic obstructive pulmonary disease, deep vein thrombosis and blood coagulation disorders. Furthermore, co-administration of Bromelain with other chemotherapeutic agents enables those agents to more easily penetrate into the tumour and hence be even more efficacious. Papain has therapeutically relevant activity in treating some mucin-producing cancers and other conditions. Other mucin effective proteases would be expected to have similar activities, and advantages can be gained (e.g. problems associated with their systemic delivery overcome or ameliorated) by delivering them in the local and sustained manner described herein.

The present invention provides methods for the treatment of mucin-producing cancers, pseudomyxoma peritonei, cystic fibrosis and chronic obstructive pulmonary disease (with other diseases or conditions being treatable depending on the proteases in the microspheres, as described above) in a patient. The method comprises administering to the patient a therapeutically effective amount of microspheres (e.g. the microspheres described above) containing mucin-affecting proteases loaded therein, wherein the microspheres are adapted to release the proteases in a sustained manner following administration.

As noted above, Bromelain has a number of therapeutic benefits, including anti-cancer activity, but its side effects when administered systemically have thus far precluded it from entering into clinical trials. However, microspheres containing Bromelain may be specifically targeted to areas of the body that require treatment, with a local delivery of a relatively small quantity of Bromelain (compared to that which would have been needed if systemically administered) being likely to significantly reduce those side effects. Cancers which Bromelain-containing microspheres may be effective in treating include cancers having a good blood supply, such as hepatocellular carcinoma, pancreatic cancer and colorectal cancer, as described above.

The method may include the intra-arterial delivery of the microspheres, where the microspheres are injected via a catheter which has been pre-positioned as close as possible to the tumour-feeding blood vessels (to avoid occlusion of vessels leading elsewhere). In this manner, the microspheres will be carried directly into (or very close to) the tumour, where the embolised microspheres will release the Bromelain (or other mucin-affecting proteases) at a sustained rate. Such a process is similar to that presently carried out in the transarterial chemoembolization (TACE) process noted above.

Direct injections of the loaded microspheres into the tumour (intra-lesional injection) may also be a useful method of delivery. In this manner, a relatively large quantity of the mucin-affecting proteases at a therapeutically effective dose may be delivered directly to the tumour, maximising its efficacy whilst minimising the risk of side effects associated with less targeted modes of delivery.

The method may include the intracavitary delivery of the microspheres into the cavity of a patient (e.g. into the peritoneum or pleural cavity). As described above, such a method would be especially useful for treatment of pseudomyxoma peritonei or other peritoneal cancers, or cancers involving the lungs or pleura. The microspheres or pharmaceutical compositions might also be administered to a recipient by routes including intraspinal, subcutaneous or intramuscular injection.

The term “therapeutically effective amount” as used herein, includes within its meaning a non-toxic but sufficient amount of an agent or composition for use in the present invention to provide the desired therapeutic effect. The exact amount required will vary from subject to subject depending on factors such as the species being treated, the age and general condition of the subject, the severity of the condition being treated, the particular agent being administered, the mode of administration and so forth. Thus, it is not possible to specify an exact “effective amount” applicable to all embodiments. However, for any given case, an appropriate “effective amount” may be determined by one of ordinary skill in the art using only routine experimentation.

In general, the microspheres and pharmaceutical compositions of the present invention can be administered in a manner compatible with the route of administration and physical characteristics of the recipient (including health status) and in such a way that the desired effect(s) are induced. For example, the appropriate dosage may depend on a variety of factors including, but not limited to, a subject's physical characteristics (e.g. age, weight, sex), whether the agent, composition or medicament is being used as single agent or adjuvant therapy, the progression (i.e. pathological state) of a disease or condition being treated, and other factors readily apparent to those of ordinary skill in the art. Various general considerations when determining an appropriate dosage of the agents, compositions and medicaments are described, for example, in Gennaro et al. (Eds), (1990), “Remington's Pharmaceutical Sciences”, Mack Publishing Co., Easton, Pa., USA; and Gilman et al., (Eds), (1990), “Goodman And Gilman's: The Pharmacological Bases of Therapeutics”, Pergamon Press.

The microspheres may generally be administered in an amount effective to achieve an intended purpose. More specifically, they may be administered in a therapeutically effective amount, which means an amount effective to prevent development of, or to alleviate the existing symptoms of, a target disease or condition. Determination of effective amounts is well within the capability of persons of ordinary skill in the art. For example, a therapeutically effective dose of given microspheres can be estimated initially from cell culture assays. For example, a dose can be formulated in animal models to achieve a circulating concentration range that includes the ICSO as determined in cell culture. Such information can be used to more accurately determine useful doses in humans and other mammalian subjects.

In general, the microspheres of the present invention may be administered to a patient in any amount whereby a therapeutic effect occurs. The nature of the therapeutic effect will depend on factors such as the mucin-related disease or condition being treated and the mucin-affecting protease being administered. When treating tumours, for example, the inventors believe that it is more appropriate to consider factors such as tumour volume rather than body weight in determining an appropriate dosage. For example, the average size of pancreatic tumour is estimated to be about 20 cm³±16 cm³. Concentrations of Bromelain (for example) required to have a substantial cytotoxic effect on pancreatic cells (as measured in vitro) will need to be greater than 20 μg/mL, thus the microspheres would need to deliver an amount of Bromelain sufficient to locally deliver more than 400 μg of Bromelain for a 20 cm³ tumour (noting that this is based on Bromelain alone and that, when combined with a chemotherapeutic agent, much less may be required). As noted above, the clearance rate of the protease out of the target area will also need to be factored into the calculations of the quantity and rate of the protease delivered to the tumour.

Typically, in treatment applications, the treatment may be for the duration of the disease state or condition. Further, it will be apparent to one of ordinary skill in the art that the optimal quantity and spacing of individual dosages can be determined by the nature and extent of the disease state or condition being treated, the form, route and site of administration, and the nature of the particular subject being treated. Optimum dosages can be determined using conventional techniques. It will also be apparent to one of ordinary skill in the art that the optimal course of administration can be ascertained using conventional course of treatment determination tests.

Where two or more entities (e.g. agents or medicaments) are administered to a subject “in conjunction”, they may be administered in a single composition at the same time, or in separate compositions at the same time, or in separate compositions separated in time, either before or after one another.

Certain embodiments of the present invention may, for example, involve administration of the microspheres or pharmaceutical compositions in multiple separate doses. Accordingly, the methods for therapeutic treatment described herein encompass the administration of multiple separated doses to a subject over a defined period of time. In some embodiments the methods may include administering a priming dose, which may be followed by a booster dose. In some embodiments, the microspheres or pharmaceutical compositions may be administered at least once, twice, three times or more.

A therapeutically effective dose refers to that amount of the microspheres (and mucin-affecting protease) required to ameliorate symptoms and/or prolong the survival of the subject under treatment. Toxicity and therapeutic efficacy of the enzymes etc. can be determined by standard pharmaceutical assays in cell cultures, and/or experimental animals (e.g. by determination of the LD50 (the dose lethal to 50% of the population) and the ED50 (the dose therapeutically effective in 50% of the population)). The dose ratio between toxic and therapeutic effects is the therapeutic index which can be expressed as the ratio between LD50 and ED50. Agents, compositions and medicaments which exhibit high therapeutic indices are preferred. The data obtained from such cell culture assays and/or animal studies may be used to formulate a range of dosage for use in humans or other mammals. The dosage of such compounds lies preferably within a range of circulating concentrations that include the ED50 with little or no toxicity. The dosage may vary within this range depending upon the dosage form employed and the administration route utilised. The exact formulation, route of administration and dosage can be selected without difficulty by an individual physician in view of the subject's condition (see, for example, Fingl et al., (1975), in “The Pharmacological Basis of Therapeutics”, Ch. 1 p. 1, which is incorporated herein by reference).

The present invention may be used to treat any suitable patient or subject. In some embodiments, the patient is a mammalian subject. Typically, the patient will be a human patient, although other subjects may benefit from the present invention. For example, the subject may be a mouse, rat, dog, cat, cow, sheep, horse or any other mammal of social, economic or research importance.

### EXPERIMENTAL RESULTS

Experiments conducted by the inventors demonstrating that mucin-affecting protease in the form of Bromelain and Papain can be loaded into commercially available microspheres and subsequently elute in a sustained manner upon exposure to physiological conditions will now be described.

### Example 1—Loading Bromelain and Papain into DC Beads® Polyvinyl Alcohol (PVA) Hydrogel Microspheres

The experiments described below were conducted using commercially available PVA hydrogel microspheres sold under the brand DC Beads® and having two different sizes: 100-300 μm and 300-500 μm. The loading of bromelain into these beads and its subsequent release was investigated.

### Experiment 1: 300-500 μm PVA Beads (DC Beads®)

100 μl of PVA hydrogel beads (DC Beads® 300-500 μm) were incubated at room temperature (23 deg C.) with vigorous agitation for 24 hrs with three solutions containing 3, 5 and 10 mg/ml bromelain, respectively. The amount of bromelain remaining in each solution after this time was analysed in order to determine how much of the bromelain had loaded into the beads.

Following this analysis, each batch of the loaded beads was gently washed in distilled water and then released into a 5 ml solution of distilled water at 37 deg C. 250 μl of each solution was removed periodically for analysis using the azo-casein assay to determine the quantity of bromelain that had been released from the beads, with the removed volume being replaced with fresh distilled water. The results of this analysis are shown in Table 1 and Graph 1, set out below.

TABLE 1

| Bromelain | Total | | | | |
|---|---|---|---|---|---|
| Soln. | loading in | | Burst release | % release | |
| (mg/ml) | 100 μl beads | % loading | % of load | with time | |
| 3.0 | 540 | 90 | 4.6 | 78 at 135 | hrs |
| 5.0 | 875 | 87.5 | 2.8 | 73 at 135 | hrs |
| 10.0 | 1750 | 87.5 | 27.4 | 100 at 25 | h |

As can be seen, once an initial burst release had occurred, a more sustained release of the remaining bromelain from the microspheres was observed, especially for the microspheres having a lower bromelain concentration. These results teach how much Bromelain can be loaded into the DC Beads, and that a higher loading increases the burst release.

### Experiment 2: 100-300 μm PVA Beads (DC Beads®)

Similar to Experiment 1, 60 μl of PVA hydrogel beads (DC Beads® 100-300 μm) were incubated with 200 μl of bromelain solution containing either 1.0 mg/ml or 3.0 mg/ml bromelain in distilled water at room temperature (23 deg C.) with agitation for 24 hrs.

The beads were removed from the 200 μl of bromelain solution, washed and were then added to 5 ml of distilled water at pH 7.0, whereupon elution commenced. 250 μl of this solution periodically removed for bromelain analysis as per Experiment 1, with the removed volume being replaced with fresh distilled water. The results of this analysis are shown in Table 2 and Graph 2, set out below.

The bromelain release rate is calculated using the linear increase in bromelain vs time (graphical) after the first 30 minutes. The burst release was found to be relatively small in these experiments, possibly because the beads were washed before being released into the distilled water.

TABLE 2

| | 1 mg/ml | 3 mg/ml |
|---|---|---|
| | Bromelain | Bromelain |
| DC Beads ® 100-300 μm | solution | solution |
| Total load (μg) | 170 | 540 |
| Percentage Loading | 85 | 90 |
| Burst release (μg) | 5 | 11.5 |
| Bromelain Release rate (μg/hr) | 1.0 | 0.45 |
| % of total load released at 23 hrs | 36.5 | 21.1 |

The results of the experiments described in Experiment 1 and 2 evidence that relatively large quantities of bromelain can be loaded into DC Beads having different sizes, and be subsequently released in a sustained manner (albeit after an initial burst release).

### Experiment 3—Determining the Durability and Proteolytic Activity of Bromelain Eluted from PVA Hydrogel DC Beads® (300-500 μm)

Bromelain was loaded into DC Beads® (300-500 μm) using the following method. The PVA hydrogel beads (80u1) were first washed in 1.0 ml of distilled water and then immersed in 200 μl of bromelain solution (1.0 mg/ml at pH.3.7) with vigorous agitation over 24 hrs at 23 deg C. A 100 μl aliquot of the bromelain solution was then analysed for bromelain proteolytic activity using the azocasein assay. The result indicated that a total of 197 μg (almost 100%) of the bromelain was loaded into the microspheres.

The so-loaded bromelain was then eluted from the DC Beads® PVA beads in the following manner. The bromelain loaded beads were removed carefully and immersed in 5.0 ml of distilled water (pH.7.0) in a 50 ml centrifuge tube. The centrifuge tube containing the beads was immersed in water bath at 37 deg .C with continuous agitation. Periodically at ½, 1, 2, 4, 6, 8 etc. hrs, 250 μL of solution was withdrawn for bromelain analysis. The lost volume (250 μL) was replaced each time with equal volume of pH 7.0 adjusted distilled water. The results are shown below in Graph 3.

As can be seen from Graph 3 shown above, Bromelain was released from the beads for almost 77 hours, after which there was no proteolytic activity as assayed by the azocaesin assay. This may indicate that either the remaining bromelain was locked within the hydrogel beads or that it was still being released, but that it had lost its proteolytic activity, possibly since it was at 37 deg C. for more than 76 hours or due to the agitation. Nevertheless, bromelain is relatively sensitive to heat, and maintaining proteolytic activity for almost 80 hours at 37 deg C. was surprising to the inventors.

Within this period about 37% (66 μg) of bromelain that was loaded into the microspheres was released in active form (i.e. having proteolytic activity).

Similar procedures were carried out for a 3.0 mg/ml bromelain solution, where 60 μl of DC Beads® (300-500 μm) were immersed in 200 μl of the bromelain solution. The results of this experiment are shown below in Graph 4.

As can be seen, when the 60 μl of DC Beads® (300-500 μm) were immersed in 200 μl of 3.0 mg/ml bromelain solution for 24 hrs, it was able to load 288 μg of bromelain (48%). Elution studies similar to those of Experiment 3 showed that active bromelain (as characterised using the azocasein assay) eluted from the microspheres for 108 hrs and the percentage bromelain that diffused out over that time was about 55% (158 ug).

### Experiment 4:—Loading Papain into Polyvinyl Alcohol Hydrogel DC Beads® (300-500 μm)

80 μL of DC Beads® (300-500 μm) were taken in a 1.5 mL centrifuge tube and washed twice with distilled water. 200 μL of a 5 mg/mL Papain solution was added and the mixture incubated at room temperature for 6 hrs with gentle agitation. The beads were then washed twice with distilled water and suspended in 5 mL PBS pH 6.5. The first sample of 250 μL was taken at 30 min, and thereafter samples were taken every hour for the next 16 hrs, with 250 μL PBS being replaced each time. The Papain concentration in the samples was measured by azo-casein assay and the cumulative release of Papain from the DC Beads as a function of time is set out in Graph shown below.

As can be seen in Graph 5, a sustained release of the Papain was achieved and continued for greater than 16 hours.

Subsequently, experiments were conducted to determine the efficacy of the Papain released from the beads. In these experiments, HT29 cells (human colorectal cancer cell line) were seeded in 24 well plates. After 24 h, the plates were treated with 5 mg/ml Papain (500 μg/80 μl beads) loaded beads in a Transwell chamber. After every 3 h, the Transwell chamber containing beads was transferred to a fresh well. This process was continued up to 3 days. These experiments showed that the Papain released from the DC Beads maintained its proteolytic activity, causing the death of all cells in the Transwell chamber after 3 hours.

### Experiment 5—Determining the Effects of Release Volume and Sampling Volume on the Release of Bromelain from DC Beads® 100-300 μm

The initial drug concentration in vivo for delivery to a tumour depends primarily on the tumour's size, with subsequent concentration of drugs in the body fluids depending on body volume (body mass). The clearance rate of drugs from the various target areas in the patient's body will depend on perfusion (blood supply), which is organ specific. The models described below (Graphs 6.0, 6.1, 6.2, 6.3 and Table 3) are intended to provide an indication of both the initial release of bromelain in the fluid, and its subsequent clearance rate.

81.0 μg of bromelain was loaded into 60 μl of PVA (100-300 μm) beads in a manner similar to that described previously. The beads were then added to 20 mL of PBS (pH 7.4, 37 degC.) and the amount of Bromelain eluted was measured as a function of time. Similar to the earlier experiments, a 250 μl of sample was withdrawn at various time points for analysis of bromelain content, with the same volume of PBS being added in order to maintain the volume of the elution media, As can be seen in the graph set out above (Graph 6.0), the release of bromelain into 20 ml of PBS (at pH 7.4) showed a burst release of 30 μg within the first ½ hour, after which a gradual release (dx/dt)=1.78 μg/hr for a period of 28 hrs was observed.

The same loaded beads were added to 5 mL of PBS (pH 7.4, 37 degC.) and the amount of Bromelain eluted was measured as a function of time, with the results shown in Graph 6.1. As can be seen, within the first 30 minutes, 8 μg was released (burst release) after which there was a slow release (linear part of the graph) over 37 hrs. Dx/dt=58/37=1.57 μg/hr.

In this model (i.e. as shown by Graph 6.1), the release media (PBS pH 7.4) was only 5.0 ml and hence the initial burst release was considerably smaller compared to the former model (graph 7.0) that uses 4 times the volume in this model. This indicates that the amount of Bromelain eluted in the burst release decreases when the volume of the release media decreases.

The same loaded beads were added to 20mL of PBS (pH 7.4, 37 degC.) and samples were taken to determine the amount of Bromelain eluted as a function of time. In these experiments, however, the sample size was 500 μL, instead of 250 μL, in order to mimic a target area in a patient's body having a higher flow/clearance rate of the drug than in the earlier experiments. As can be seen in Graph 6.2 (below), dx/dt=2.45 μg/hr in these experiments, and all 81 μg of the loaded bromelain was released from 60 ul of PVA beads within 23 hours.

In the final of this series of experiments, the same loaded beads were added to 20 mL of PBS (pH 7.4, 37 degC.) and samples were taken to determine the amount of Bromelain eluted as a function of time. In these experiments, however, the sample size was 1 mL, instead of 500 μL or 250 μL, in order to mimic a target area in a patient's body having an even higher flow/clearance rate of the drug. The results of these experiments are shown above in Graph 6.3. As can be seen, compared to all the other release models used, this has the highest sampling volume (representing high flow/clearance) and hence, the complete release of 81 μg of loaded bromelain was released within 17 hrs (dx/dt=4.5 μg/hr).

TABLE 3
Summary of Graphs 6.0, 6.1, 6.2 and 6.3

| Release | | Sampling | BR release | Total release | Burst release |
|---|---|---|---|---|---|
| vol (ml) | | vol (ml) | rate μg/hr | time (hr) | (μg) |
| 5 | ml | 0.250 | 1.57 | 42 | 9 |
| 20 | ml | 0.250 | 1.78 | 28 | 30 |
| 20 | ml | 0.500 | 2.45 | 22 | 30 |
| 20 | ml | 1.0 | 4.5 | 11 | 30 |

In vivo, choice of release rate volume will depend on a number of factors, including body volume, metabolic rate, organ perfusion, pH, site at which beads are delivered etc. The clearance rate of the eluted mucin-affecting protease in vivo will be an important factor in determining a suitable dosage regimen. The experiments described here have been performed to simulate increased flow at the target area and demonstrate that the choice of release volume will influence the magnitude of initial burst, and that the sampling volume (i.e. the clearance rate in vivo) will influence the subsequent release rate (see graph 7).

### Experiment 6—Determining the Effects of pH in Bromelain Loading Solution on the Release Profile of the Microspheres

In these experiments, DC Beads® 300-500 μm, were loaded with bromelain solutions having different pH levels of 2.5, 3.4 and 4.0. The bromelain loaded DC Beads® were then added to 5.0 ml of PBS (at pH 6.5) with a 250 μl sampling volume being removed at specific intervals and replaced with an equal volume of fresh PBS. The results of these experiments are shown in Graph 8 and Table 4.

TABLE 4

| | Burst release | | | | Loading |
|---|---|---|---|---|---|
| pH | (½ hr) | | dx/dt (μg/hr) | Total loading | efficiency (%) |
| 2.5 | 7.5 | μg | 5.24 | 575 | 96 |
| 3.4 | 150 | μg | 22.28 | 573 | 95.5 |
| 4.0 | 100 | μg | 20.3 | 571 | 95 |

This experiment indicates that the pH of the loading solution does have an effect on the burst release and the subsequent rate of bromelain release. Whilst the loading was very similar at all three pH levels, the release rate was considerably smaller at loading pH of 2.5 whilst that at pH 3.4 and pH 4.0 were quite similar.

### Experiment 7—Determination of pH Effect on the Loading and Release of Bromelain from DC Beads® PVA Hydrogel (100-300 μm)

DC Beads® 100-300 μm (60 ul) were loaded with bromelain (3.0 mg/ml) prepared in either water (pH 2.8, 3.0 &3.2) or in PBS (pH 2.77) by adding 200 μl of the solution with agitation on a shaker at ambient room temperature (25 deg C.) over 24 hrs. The bromelain solution was then carefully removed using a pipette and analysed for residual bromelain in order to determine the total load in the beads. Results of this experiment are tabulated below in Table 5.

The PVA beads were then added to 10 ml of PBS (pH 6.5) with gentle agitation in a water bath at 37 deg C. To determine burst release of the bromelain, at 0.5 hr, 500 μl of solution was removed for analysis (with 500 μl of fresh PBS being added to maintain a constant volume). Then after at hourly intervals, a similar procedure was carried out for analysis of bromelain. These experimental results are shown below in Graph 9 and Table 5.

TABLE 5

| | | | | Loading |
|---|---|---|---|---|
| Loading in H₂0 | | | | in PBS |
| | pH 2.8 | pH 3.0 | pH 3.2 | pH 2.77 |
| Total loading (μg) | 535 | 525 | 518 | 594 |
| % loading | 89 | 87.5 | 86.3 | 99 |
| Burst (μg) | 102 | 110 | 117.35 | 114.79 |
| Burst (% of total load) | 19 | 21 | 23 | 19 |

Rate of bromelain release

| | | | | |
|---|---|---|---|---|
| dx/dt (μg/hr) | 17.58 | 18.7 | 19.41 | 19.7 |
| 0-12 hrs | | | | |
| dx/dt | 6.16 | 6.16 | 6.17 | 6.75 |
| 13-24 hrs | | | | |
| dx/dt | 4.75 | 4.33 | 4.41 | 5.17 |
| 25-36 hrs | | | | |
| dx/dt | 3.5 | 3.25 | 3.16 | 3.83 |
| 37-48 hrs | | | | |
| TOTAL % of BR | 75 | 77.5 | 80 | 75 |
| Release at 48 hrs | | | | |

The percentage of loading is almost 100% using bromelain solubilised in PBS at pH 2.77, indicating that it may be a good method to use for efficient bromelain loading. At the same time, bromelain in water at the pH investigated is also quite efficient, the loading efficiency varying from 86-89%.

Burst release is lowest with loading of bromelain in water at pH 2.8 as compared to the other loading pH levels or in PBS (pH 2.77). However, the burst release when compared using % of total load, both loading in water at pH 2.8 and PBS at pH 2.77 seems to perform equally.

### Experiment 8—Determination of How the pH of the Loading PBS Affects the Loading and Release of Bromelain from DC Beads® PVA Hydrogel (100-300 μm)

Bromelain (3.0 mg/ml) was prepared in separate samples of PBS at pH 2.0, 2.2, 2.4 & 2.6. 60 μl of DC Beads® (100-300 um) was added to 200 μl of each bromelain solution and placed on an agitator at ambient room temperature (23 deg C.) for 24 hours. 200 μl of the supernatant solution of each sample was carefully removed and analysed using the azo-casein assay for residual bromelain content in order to quantify the amount of bromelain absorbed by the beads.

The beads were all added to 10 ml of PBS (at pH 6.5), and 500 μl of the solution was collected at 30 minutes to evaluate burst release (with that volume being replaces with 500 μl fresh PBS), and thereafter at 1 hour, 2, 3 etc. The bromelain eluted in the 500 μl of PBS was quantified over a time period until there was no bromelain release (azocaesin proteolytic activity). The results of these experiments are shown in the graphs and table set out below (Graph 10, Tables 6.0, 6.1, 6.2). It is assumed that the more acidic the loading solution, the more prolonged release will be (compare to graph 8).

The rate of bromelain release after the burst was divided into periods of 12 hours and the rate of bromelain elution was calculated from the linear part of the graph vs time.

TABLE 6.0
Total loading and burst release:

| | BR loading in | % BR | Burst release | Burst release |
|---|---|---|---|---|
| pH | PVA beads (μg) | loaded | (μg) | (% of total load) |
| 2.0 | 445 | 74 | 15 | 3.4 |
| 2.2 | 525 | 87.5 | 30 | 5.7 |
| 2.4 | 525 | 87.5 | 62 | 12 |
| 2.6 | 525 | 87.5 | 120 | 23 |

TABLE 6.1
Rates of release of Bromelain over time

| | dx/dt | dx/dt | dx/dt | dx/dt | dx/dt |
|---|---|---|---|---|---|
| | (1-12 h) | (13-24 h) | (25-36 h) | (37-48 h) | (49-60 h) |
| pH | μg/hr | μg/hr | μg/hr | μg/hr | μg/hr |
| 2.0 | 1.32 | 1.29 | 1.24 | 2.27 | 1.34 |
| 2.2 | 1.32 | 1.36 | 1.33 | 1.37 | 1.36 |
| 2.4 | 2.79 | 2.58 | 2.6 | 2.41 | 2.13 |
| 2.6 | 5.33 | 4.6 | 3.8 | 3.2 | 2.75 |

TABLE 6.2
Percentage release of total bromelain over time

| pH | 12 hrs | 24 hrs | 36 hrs | 48 hrs | 60 hrs |
|---|---|---|---|---|---|
| 2.0 | 7.3 | 11 | 15 | 18.7 | 22.6 |
| 2.2 | 9.0 | 12.4 | 15.7 | 19 | 22.6 |
| 2.4 | 18.8 | 25.33 | 32 | 38 | 43 |
| 2.6 | 36.3 | 47.7 | 57 | 65 | 72 |

As can be seen, the pH at which the Bromelain is loaded into the microspheres affects the amount that can be loaded, the burst release and the subsequent elution rate of the loaded Bromelain. The pH of the loading media may therefore be utilised to adjust the elution properties of the microspheres to suit specific patients and treatment regimens.

For example, the average size of a pancreatic tumour is estimated to be about 20 cm³±16 cm³. Using 60 μl of DC Beads® 100-300 μm loaded with bromelain (3.0 mg/ml) at pH 2.6, the burst release of 120 μg would essentially give the tumour a bromelain concentration of 120/20 =6 μg/ml. However, a concentration greater than 20 μg/ml bromelain alone is required to have substantial cytotoxic effect as a single agent on the tumour cell (PANC-1 cells has an IC50 of 18 μg/ml and IC75 of 50 μg/ml in in vitro studies). In order to increase the concentration of bromelain to 60 μg/ml for a 20 cm³ tumour therefore, 600 μl of loaded beads would be required.

After the burst release, 60 μl of DC Beads® 100-300 um releases 5.33 μg/hr and a 10 fold increase in the volume of beads (600 μl) would essentially release 53.3 μg/hr for the first 12 hours. Assuming clearance to be 53.3 μg/hr, then a steady state of 60 ug/ml of bromelain can be maintained for at least 12 hours after which, the quantity released declines approximately by 0.007% every hour.

A 80 kg lean patient may have a blood volume of around 6 litres which means that 53 μg/hr of bromelain released will be diluted to about 8.83 ng/ml and of which a great portion binds to albumin, antitrypsin and macroglobulin. Toxicity on blood coagulation parameters may be a problem however, this is with flow and does not represent an upstream embolization model, such as that in liver and pancreatic cancers. Lower exposure to bromelain to be used in a synergistic model with chemotherapy would also be possible.

Loading at pH 2.4 may also be used for this tumour model with probably a 20 fold increase in volume of loaded PVA beads used to simulate a similar scenario as in the previous example.

### Example 2: HEPASpheres Sodium Acrylate Alcohol Copolymer 30-60 um

### Experiment 9—Loading and Release of Bromelain from HEPASphere Microsphere (30-60 μm) in PBS (3.0 mg/ml) at Different pH

HEPA microspheres (40 μl) were treated to 300 μl of bromelain solution (3 mg/ml) in PBS at different pH (2.0, 2.2, 2.4 & 2.6) with continual agitation for 24 hours. The tubes containing the beads and the solution were then centrifuged, and the supernatant (300 μl) was aspirated and analysed for residual bromelain content in order to quantify the loading of bromelain.

The HEPA beads were then added to 10 ml of PBS (at pH 6.5) in a 50 ml centrifuge tube that was immersed in a water bath at 37 deg C. with continuous agitation. 500 μl of solution was removed starting at ½ hr and subsequently every hour. The removed sampling solution (500 ul) was replaced with each sampling. The sampling solution was then analysed for bromelain content using the azocasein assay. The results of these experiments are shown below in Graph 11 and Tables 7, 7.1 and 7.2.

TABLE 7.0
Total loading of microsphere beads:

| Loading pH | Loading (total) μg | % loading | Loading/μl beads (μg/μL) |
|---|---|---|---|
| 2.6 | 650 | 72 | 16.25 |
| 2.4 | 650 | 72 | 16.25 |
| 2.2 | 700 | 78 | 17.5 |
| 2.0 | 700 | 78 | 17.5 |

TABLE 7.1
Burst release

| | | Burst Release (as a % |
|---|---|---|
| Loading pH | Burst Release (μg) | of total loading) |
| 2.6 | 125 | 19.2 |
| 2.4 | 62 | 9.5 |
| 2.2 | 30 | 4.3 |
| 2.0 | 15 | 2.14 |

The release rate every 12 hrs (dx/dt) was calculated using the linear part of the graph, excluding the burst quantity of bromelain.

TABLE 7.2
Release rate/hr

| | | | | dx/dt 37-48 |
|---|---|---|---|---|
| Loading pH | dx/dt 1-12 | dx/dt 13-24 | dx/dt 25-36 | (μg/hr) |
| 2.6 | 10.1 | 7.58 | 7.0 | 6.9 |
| 2.4 | 5.1 | 4.58 | 4.42 | 4.42 |
| 2.2 | 3.0 | 2.68 | 2.68 | 2.62 |
| 2.0 | 1.42 | 1.33 | 1.3 | 1.3 |

The beads (40 μl) were all exposed to bromelain solution (300 μl) containing 900 μg of bromelain and there is only a slight difference in loading capacity at the different pH levels, although at 2.2 and 2.0 pH, the loading is similar and slightly higher than that of at pH 2.6 or 2.4. In other experiments (not described) the loading capacity was found to be much higher (87%) when exposed to 1200 μg of bromelain at pH 3.4.

The burst release was the highest (125 μg-19.2% of the total loading) when the bromelain was loaded at pH.2.6, and lowest when the bromelain was loaded at pH 2.0. pH is known to have an effect on the pore size of the microspheres, and hence this may have influenced the burst release.

The release rate that was calculated over every 12 hrs shows that at pH 2.6, it was the highest again, indicating that there may be a bearing on the loading pH and the pore size of the beads at the particular pH. At 52 hours, HEPA microspheres loaded at pH 2.6 had eluted 86% of the loaded bromelain, whilst loading at pH 2.4, it was 48%, loading at pH 2.2 and 2.0 they were 26 and 13% respectively. This indicates that loading pH possibly plays a crucial role in the release pattern and total release at a particular time.

HEPA microspheres are polyvinyl alcohol-co-sodium acrylate and, in an acidic environment, the protonated amine group with a positive charge in the bromelain forms a linkage with the acetate group with a negative charge and this is the basis of tethering bromelain to the beads. When the loaded beads are added to the release medium, the ionic bonds break easily to release the bromelain, until such time an equilibrium is attained between the amounts of bromelain tethered and free bromelain in the PBS at pH 6.5. On sampling and replenishment with fresh PBS solution, there is a drop in Bromelain concentration, which results in a further release of bromelain from the beads until equilibrium is achieved. In the current model, 10 ml of PBS was used, with removal of 500 μl for sampling and this amounts to a flux change of 5% or a clearance of 5%.

The mean liver tumour size is reported to be 21.8 cc (Dachman et al Tumor size on Computed Tomography Scans. Cancer, 2001; 91(3):555-560) and for unresectable tumours, treatment with bromelain loaded microspheres that are capable of delivering a concentration of around 20 μg/ml is required. 40 μl of microspheres loaded at pH of 2.6 have a burst release of 125 μg bromelain and this translates to 5.73 μg/cc at the tumours. Delivering 160 μl of microspheres to the site of the tumours will essentially increase the bromelain concentration to 22.9 μg/cc. Assuming clearance to be around 10%/hr, that equates to 2.3 μg/hr is lost. The release rate/hr for the first 24 is about 8 μg/hr (X4=32 μg/hr) and this would offset the lost bromelain at the tumour site.

### Example 3—TANDEM Microspheres 75 μm

### Experiment 10—Loading and Release of Bromelain from TANDEM 75 μm Microspheres

40 uL Tandem beads (75 μm) were collected in a 1.5 mL centrifuge tube. 200 uL of 5 mg/mL bromelain in distilled water was added. The tube was left for 24 hrs at room temperature with gentle agitation. Next day, the beads were washed twice with distilled water and resuspended in 5 mL distilled water. First, a 250 μL sample was collected at 30 mins and subsequent samples collected thereafter every hour for the next 32 hrs (with 250 μL of fresh water being replaced each time). The Bromelain content of each sample was measured by azo-casein assay, and used to calculate the release profile of the bromelain as shown in Graphs 12.1 and 12.2.

No more Bromelain was released after 32 hrs. These experiments demonstrate that Bromelain can be loaded into another form of commercially available microspheres and subsequently eluted in a still-active form and in a sustained manner. The release pattern of bromelain in TANDEM spheres might be modified by adjusting size of sphere, pH when loading and amount loaded, coating, and other techniques described earlier.

### Example 4—Coated DC Beads® PVA Hydrogel (300-500 μm) Microspheres

### Experiment 11—Loading and Release of Alginate Coated DC Beads® PVA

DC Beads (300 uM-500 μM) were loaded with bromelain (5 mg/mL) at room temperature for 24 hrs. The bromelain loading in the beads was calculated to be 900 μg. The beads were then washed twice with distilled water before being immersed in a 2% alginate solution and then dipped in 2% CaCl solution for 15 min in order to form an alginate coating over the outermost surface of the microsphere.

The bromelain-containing, alginate coated beads were then added to 10 mL water and their bromelain release was measured in the manner described above for the next 30 hrs, with the results being shown in Graph 13.2. Bromelain loaded DC Beads® 300-500 μm (uncoated) were used as control (Graph 13.1).

As can be seen from the graphs set out above, the bromelain contained within the alginate coated DC Beads® eluted at a much slower rate, with only about 10% of the total bromelain having eluted from the coated bead in about 10 hours, compared to about 66% for the non-coated microspheres. There was also a reduced burst effect in the alginate coated beads.

### Example 5—DC Beads® Microspheres Co-Loaded with Bromelain and Doxorubicin

Three batches of DC Beads® (300 um-500 um) were prepared in a manner similar to that described previously. The first batch of the DC Beads® were loaded with bromelain (1 mg/mL) alone and the second batch were loaded with Doxorubicin 0.25 mg/mL alone. The third batch were first loaded with lmg/ml bromelain for 24 hrs and then, the next day, loaded with 0.25 mg/mL doxorubicin for 6 hrs.

CFPAC-1 cells (human pancreatic cancer cell line) were plated in 96 well plates. Serial dilutions of the beads were made and deposited in quadruplet wells and plated incubated for 72 hrs. At end of incubation period, SRB assays was done and the number of beads per well counted, with the results being tabulated as shown below (graphs 14.1, 14.2, 14.3).

In Graph 14.1, there is a dose (bead dilution) effect with growth inhibition at 40 beads/well and 22 beads/well, but the effect is evidently lost at 9 beads/well.

As can clearly be seen from the experimental results presented above (graphs 14.1, 14.2, and 14.3), more cells were killed with beads loaded with bromelain and doxorubicin as compared to bromelain or doxorubicin alone loaded beads. These data are indicative of a synergy between doxorubicin and bromelain compared to either doxorubicin or bromelain alone.

### Example 6—The Efficacy of Bromelain-Containing DC Beads® 100-300 um Microspheres

DC Beads (100-300 μM) were loaded with Bromelain (400 μg/mL) in a manner similar to that described above. The microspheres were subsequently serially diluted and their efficacy against CFPAC-1 cells were determined as described above in Example 5. The results of these experiments are set out below in Graphs 15.1 and 15.2.

These data (Graph 15.1, above) shown that inhibition was reduced at less than 62 beds/well in pancreatic cancer cell line CFPAC-1 with 0.149 ug/bead. 130 beads were required to achieve 90% cell death at this loading dose.

In the pancreatic cancer cell line CFPAC-1, inhibition was reduced at less than 22 beads/well for the more heavily loaded beads (Graph 15.2, below). Graph 15.1 shows that 130 of the less-heavily bromelain loaded beads (0.149 ug/bead) were required to kill 90% of cells, whereas only 43 of the more-heavily loaded beads (0.403 ug/bead) were required to achieve roughly the same efficacy in graph 15.2.

In this experiment, the inhibitory effect of different concentrations of bromelain (0.149 or 0.403 μg/bead) loaded beads was tested. The data showed that the higher the concentration of bromelain loaded per DC Bead the lower number of beads are required to inhibit cell proliferation.

### Example 7—Time-Point Studies to Evaluate the Length of Exposure Needed

OVCAR-3 cells of a human ovarian cancer cell line were seeded into 96 well plates. After 24 hours, the plates were treated with doxorubicin (50 nM), N-acetylcysteine (2.5 mM) and differing concentrations of bromelain (not administered in microspheres). After 1 hour, 3 hours, 6 hours, 18 hours, 24 hours and 48 hours, the drugs and media were removed and the plates were washed with PBS. Doxorubicin treatment was recommenced in the appropriate wells and drug free media was added to all other wells. All plates were treated for a further 72 hours. The results of these experiments are shown below in graphs 16.1, 16.2 and 16.3.

The purpose of these studies was to determine the length of time that bromelain would need to be eluted from microspheres in order to have a synergistic effect with a co-administered chemotherapeutic agent. These results indicate that on exposure to bromelain of 24 hours, and preferably 48 hours, good synergy with doxorubicin at 50 nM was seen. At higher concentrations of doxorubicin, such as 100 nM (results not shown), even exposure to bromelain for 3 hours was observed to produce a synergistic cancer cell killing effect.

### Example 8—Pre-Clinical Animal Studies of Bromelain and Doxorubicin Loaded DC Beads®

DC Beads (100-300 μM) were loaded with Bromelain in a manner similar to that described above.

In this safety study, New Zealand rabbits were treated with DC beads that had been loaded with a total of 5 or 10 mg Bromelain in a manner similar to that described above. The suspension of beads was injected directly into the common hepatic artery (i.e. via an intra-arterial route), whereupon the DC beads were carried by the blood flow until they embolised and eluted the Bromelain over time.

Post-treatment, at 1 h, 3 h, 6 h, 24 h or 7 d, animals were euthanized. Then, post-mortem, comparative observations of the internal organs, bromelain concentration measurements in plasma and liver were performed. These observations are set out below in Graphs 17.1-17.6.

As can be seen from the graphs set out above, the bromelain contained within the DC Beads® eluted in the liver over about 24 hours (Graphs 17.1, 17.2 and Graphs 17.5) after intra-common hepatic artery injection. Minimal amount of bromelain could reach the blood stream up to 6 hours (Graphs 17.3, 17.4 and Graphs 17.6). Gross examination of the livers showed distribution of beads in the targeted liver lobes. Recovery of normal tissues was observed at 7 d post-treatment (results not shown). Briefly, the results of this study showed the safety of DC beads loaded with bromelain.

### Example 9—Cytotoxic Activity of DC Bead (100-300 μm) Loaded with Bromelain on Cancer Cells (ASPC-1 and HT-29)

Cells of ASPC-1 and HT-29 cell lines were seeded into 96 well plates. After 24 hours, plates were treated with 5 mg/ml Bromelain loaded into DC Beads (100-300 μM ) in a manner similar to that described above (see Example 5). The microspheres were subsequently serially diluted. After 48 hours, the drugs and media were removed and cell proliferation was tested using SRB assay. The results of these experiments are shown below in Graphs 18.1 and 18.2.

In this experiment, the inhibitory effect of a serial dilutions of bromelain loaded beads (50, 30 and 10 beads/well) was tested. The data shown that dilutions of Bromelain loaded DC Beads were more effective in the colorectal cell line HT-29 (Graph 18.2) compared to the pancreatic cancer cell line ASPC-1 (Graph 18.1).

### Example 10—Duration of Activity and Cytotoxic Activity of DC Beads (300-500 μm) Loaded with Bromelain on the Pancreatic Cancer Cells (CFPAC-1)

Cells of the pancreatic cancer CFPAC-1 cell lines were seeded into 24 Transwell plates. After 24 hours, plates were treated with Bromelain loaded into DC Beads (300-500 μM) using Transwell chamber inserts. After every 3 hours, Transwell inserts containing the beads were transferred to another fresh wells. This process was continued for 3 days. Cell proliferation was tested using SRB assay. The results of these experiments are shown below in graph 19.1.

In this experiment, the results showed that beads were releasing cytotoxic doses of Bromelain up to 17 hours.

### Example 11—Potential Method of Treating Cancer

The following example describes how the inventors believe that microspheres in accordance with an embodiment of the present invention may be used to treat a cancerous tumour, for example in treating primary or secondary liver cancers. The process is similar to that of the TACE process described above, where the microspheres are injected into an artery feeding a cancerous tumour. The microspheres are carried in the patient's artery until they become physically trapped, before the arterial bed is reached. In this manner, the microspheres cut off or limit the tumour's blood supply and locally deliver the bromelain (or other mucin-affecting proteases) and any other co-loaded or co-administered further agents in a sustained manner.

As described herein, the present invention provides a novel delivery vehicle via which effective amounts of Bromelain (or other mucin-affecting proteases which have therapeutic applications) are deliverable to a patient in a manner whereby its potential side effects were minimised. Embodiments of the present invention provide a number of advantages over existing therapies, some of which are summarised below:

microspheres of the present invention provide a delivery method for the mucin-affecting protease that is local and which provides a sustained release of the protease (optionally with other actives), enhancing its effect whilst reducing potential side effects;

the present invention can result in high local concentrations of mucin-affecting proteases at a target area, with all the attendant benefits of such, but without the risks associated with systemic toxicity;

the present invention may improve penetration of drugs into cancers (especially tumours with fibrous coats or surrounded in adhesions), and may provide synergistic effects when used with other chemotherapeutic agents; and

the sustained release of mucin-affecting proteases can be engineered to occur over the reproduction time of the relevant cells, ensuring cell death.

It will be understood to persons skilled in the art of the invention that many modifications may be made without departing from the spirit and scope of the invention. All such modifications are intended to fall within the scope of the following claims.

It will be also understood that while the preceding description refers to specific forms of the microspheres, pharmaceutical compositions and methods of treatment, such detail is provided for illustrative purposes only and is not intended to limit the scope of the present invention in any way.

It is to be understood that any prior art publication referred to herein does not constitute an admission that the publication forms part of the common general knowledge in the art.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A microsphere for delivery to a target area in a patient's body, the microsphere:
containing a mucin-affecting protease loaded therein, and
adapted to elute the mucin-affecting protease in a sustained manner when exposed to physiological conditions.

2. The microsphere of claim 1, wherein the microsphere comprises a hydrogel into which the mucin-affecting proteases are loaded.

3. The microsphere of claim 1 or claim 2, wherein the microsphere comprises a polyvinyl alcohol hydrogel, a poly(vinyl alcohol-co-sodium acrylate) hydrogel, a hydrogel network of poly(ethylene glycol) and 3-sulfopropyl acrylate, poly(lactic-co-glycolic acid) or polylactic acid-containing hydrogels or a hydrogel core consisting of sodium poly(methacrylate) and an outer shell of poly(bis[trifluoroethoxy]phosphazene).

4. The microsphere of any one of claims 1 to 3, wherein the microsphere comprises an outer coating.

5. The microsphere of any one of claims 1 to 4, wherein the microsphere comprises an alginate outer coating.

6. The microsphere of any one of claims 1 to 5, wherein the microsphere has a diameter of between about 30 and about 700 micrometres.

7. The microsphere of any one of claims 1 to 6, wherein the microsphere is adapted to elute the mucin-affecting protease over a period of time of between about 5 hours to about 120 hours.

8. The microsphere of any one of claims 1 to 7, wherein the microsphere is adapted for delivery to the patient intra-arterially, intralesionally, intra-abdominally or intracavitarily.

9. The microsphere of claim 8, wherein the microsphere is adapted to be delivered to the patient's peritoneum or pleural cavity.

10. The microsphere of any one of claims 1 to 9, wherein the mucin-affecting protease is selected from one or more of the group consisting of plant derived proteases, fungal proteases and bacterial proteases.

11. The microsphere of claim 10, wherein the plant derived protease is selected from one or more of the group consisting of Bromelain, Papain, Ficain, Actinidain, Zingibain and Fastuosain.

12. The microsphere of any one of claims 1 to 11, wherein the microsphere contains a further agent.

13. The microsphere of claim 12, wherein the further agent is selected from one or more of the group consisting of a chemotherapeutic agent, a radiotherapeutic agent, a mucolytic agent and a contrast agent.

14. The microsphere of claim 12 or claim 13, wherein the further agent is a chemotherapeutic agent selected from one or more of the group consisting of gemcitabine, paclitaxel, docetaxel, doxorubicin, irinotecan, mitomycin C, oxaliplatin, carboplatin, 5-fluorouracil and cisplatin.

15. A pharmaceutical composition comprising:
microspheres for delivery to a target area in a patient's body, the microspheres containing a mucin-affecting protease loaded therein and being adapted to elute the mucin-affecting protease in a sustained manner when exposed to physiological conditions; and
a pharmaceutically acceptable carrier.

16. A pharmaceutical composition comprising the microspheres of any one of claims 1 to 14 and a pharmaceutically acceptable carrier.

17. A method for loading a mucin-affecting protease into microspheres, the method comprising:
adding the microspheres to a solution having an acidic pH and, optionally, an ionic strength similar to that at a target area in a patient's body;
mixing the solution comprising the microspheres with a solution comprising the mucin-affecting protease;
agitating the mixture for a time sufficient for the mucin-affecting protease to be loaded into the microspheres.

18. The method of claim 17, wherein the pH of the solution is between about 2 and about 6.

19. A method for the treatment of a mucin-producing cancer, pseudomyxoma peritonei, cystic fibrosis or chronic obstructive pulmonary disease, the method comprising:
administering to a patient a therapeutically effective amount of microspheres containing a mucin-affecting protease loaded therein, wherein the microspheres are adapted to elute the mucin-affecting protease in a sustained manner following administration.

20. A method for the treatment of a mucin-producing cancer, pseudomyxoma peritonei, cystic fibrosis or chronic obstructive pulmonary disease, comprising administering a therapeutically effective amount of the microspheres of any one of claims 1 to 14 or a pharmaceutical composition of claim 15 or 16 to a patient in need thereof.

21. The method of claim 19 or claim 20, wherein the therapeutically effective amount of the microspheres containing mucin-affecting proteases loaded therein are administered to the patient intra-arterially, intralesionally, intra-abdominally or intracavitarily.

22. The method of any one of claims 19 to 21, further comprising co-administering a therapeutically effective amount of a further therapeutically effective agent.

23. The method of claim 22, wherein the further therapeutically effective agent is selected from one or more of the group consisting of a chemotherapeutic agent, a radiotherapeutic agent, a mucolytic agent and a contrasting agent.

24. The method of claim 22 or claim 23, wherein the further therapeutically effective agent is co-administered within the same microspheres as those containing the mucin-affecting proteases.

25. The method of claim 22 or claim 23, wherein the further therapeutically effective agent is co-administered separately from the microspheres containing the mucin-affecting proteases.

26. The method of claim 25, wherein the further therapeutically effective agent is co-administered simultaneously or sequentially from the microspheres containing the mucin-affecting proteases and, when sequentially, either before or after the microspheres.

27. The method of any one of claims 19 to 26, wherein the mucin-producing cancer is selected from the group consisting of liver cancer (primary or secondary), pancreatic cancer, lung cancer, thyroid cancer, stomach cancer, cancer of the appendix, peritoneal cancer, hepatocellular cancer, prostate cancer, breast cancer, colorectal cancers, ovarian cancers, mesothelioma, neuroblastoma, small bowel cancer, lymphoma and leukaemia.

28. Use of the microspheres of any one of claims 1 to 14 for the manufacture of a medicament for the treatment of a mucin-producing cancer, pseudomyxoma peritonei, cystic fibrosis or chronic obstructive pulmonary disease.

29. Use of the microspheres of any one of claims 1 to 14 for the treatment of a mucin-producing cancer, pseudomyxoma peritonei, cystic fibrosis or chronic obstructive pulmonary disease.

30. The microspheres of any one of clams 1 to 14 for use as a medicament.

31. The microspheres of any one of clams 1 to 14 for use in the treatment of a mucin-producing cancer, pseudomyxoma peritonei, cystic fibrosis or chronic obstructive pulmonary disease.

32. A composition comprising microspheres into which a mucin-affecting protease has been loaded, the microspheres being adapted to elute the mucin-affecting protease in a sustained manner when exposed to physiological conditions.

33. An injectable composition comprising microspheres into which a mucin-affecting protease has been loaded, the microspheres being adapted to elute the mucin-affecting protease in a sustained manner when exposed to physiological conditions.

34. A sustained release formulation comprising microspheres into which a mucin-affecting protease has been loaded, the microspheres being adapted to elute the mucin-affecting protease in a sustained manner when exposed to physiological conditions.
